(19) United States
(12) Patent Application Publication
(10) Pub. No.: US 2026/0263404 A1
(43) Pub. Date: Sep. 10, 2026

(54) PRODUCTS, METHODS, AND SYSTEMS FOR IMPLANTABLE MEDICAL DEVICES

(71) Applicant: SterileCare, Inc., Markham (CA)

(72) Inventor: Karen Bernice Mueller, Holland Landing (CA)

(21) Appl. No.: 19/556,222

(22) Filed: Mar. 4, 2026

Related U.S. Application Data
(60) Provisional application No. 63/766,912, filed on Mar. 4, 2025.

(51) Int. Cl. A61K 31/198; A61M 25/00

(52) U.S. Cl. CPC A61K 31/198; A61M 25/0017; A61M 2025/0056

(57) ABSTRACT

To reduce the risks of catheter associated bloodstream infections (CABSIs), particularly those involving multidrug-resistant organisms (MDROs), systems, compositions, products, and methods are disclosed related to reducing infection or the risks of infection associated with implantable medical devices. Such methods include the use of EDTA solutions for the management of implantable medical devices.

### CROSS-REFERENCE TO RELATED APPLICATIONS

This application claims priority to U.S. Provisional Application No. 63/766,912 titled “PRODUCTS, METHODS, AND SYSTEMS FOR IMPLANTABLE MEDICAL DEVICES,” filed Mar. 4, 2025, the entire contents of which are incorporated by reference herein.

### FIELD OF THE DISCLOSURE

The present disclosure pertains to systems, compositions, products, and methods related to reducing infection or the risks of infection associated with implantable medical devices, including infection from multidrug-resistant organisms. In particular, compositions, products, methods, and systems related to catheters and catheter lock solutions containing ethylenediaminetetraacetic acid (EDTA) and other compounds are described that may restrict microbial growth, kill microbes, eliminate or reduce biofilm, or prevent or reduce blood clot formation.

### BACKGROUND

The overuse of antibiotics in the health care industry has led to a widespread problem of multidrug-resistant organisms (MDROs), in which mutations have circumvented the antimicrobial mechanisms of commonly used drugs. The use of antibiotics in catheter lines as lock solutions may have contributed to this crisis. There is now a need for improved approaches in all aspects of healthcare to reduce the spread of MDROs and to prevent infection in patients, especially catheter-associated bloodstream infections (CABSIs), which are one of the most common causes of healthcare-associated infections.

To reduce the risk of MDRO infection and particularly MDRO-related CABSI in patients with implantable medical devices, there is a growing need for improved systems, compositions, products, and methods of managing the care of such patients. The need is great for those with implantable medical devices in contact with the bloodstream, such as central venous access devices (CVADs) or other vascular catheters, though there is a need for improved management of both non-vascular catheters, such as urinary catheters, to reduce CABSIs and to reduce the impact of MDROs. VADs may include central venous catheters (CVCs), such as those inserted into the body through the subclavian vein, jugular vein, or femoral vein (e.g., implanted ports or tunneled catheters), as well as arterial catheters, or catheters that can be inserted into one of the peripheral veins of the extremities often called peripherally inserted catheters (PICCs and PIVs).

Some catheters, such as VADs, may be present in the body for prolonged periods of time and may be used to give intravenous fluids, perform blood transfusions, or provide medications (e.g., chemotherapy or other drugs) over a period of time (e.g., greater than 3 days, 1 week, 2 weeks, 3 weeks, 6 weeks, 15 weeks, 25 weeks, one year, etc.). This long-term presence in the body poses particularly great challenges in maintaining the catheter in a safe, functional state.

With the spread of MDROs and the steady rise in the number of immune-compromised individuals, there are concerns that colonized implantable medical devices can be carriers of MDROs in communities and hospitals worldwide. There is a growing need for more effective ways to reduce such threats and improve the methods and materials used for catheters and implantable devices.

In any case, there is a need for improved catheter products and methods of using catheters and/or a need for improved products and methods of using such products that can mitigate one or more of the many challenges associated with the use of implantable medical devices. Such problems may include the “triple threat” of bacteria, biofilms, and blood clots that catheters and other implantable medical devices face, threats that are particularly severe as MDROs abound. Venous catheters such as PIVs and CVADs generally require the use of antimicrobial lock solutions (ALSs) to reduce the risk of CABSIs, but the need for improved approaches continues to grow in light of the threat of MDROs, among other concerns.

MDROs can theoretically arise from numerous bacteria. Examples of MDROs that have been detected and sometimes have posed severe challenges in healthcare systems around the world include: carbapenem-resistant gram-negative organisms, such as Carbapenem-resistant Enterobacteriaceae (CRE) or carbapenem-resistant Pseudomonas, extended-spectrum β-lactamase-producing (ESBL) producing Gram-negative bacteria, methicillin-resistant Staphylococcus aureus (MRSA), vancomycin-resistant enterococci (VRE), Candida krusei, Candida auris, Candida haemulonii, carbapenemase-producing gram-negative bacteria such as carbapenemase-producing Enterobacteriaceae (CPE), Enterococci such as Enterococcus faecium, Klebsiella aerogenes, Klebsiella pneumoniae, Citrobacter freundii, various Acinetobacter species, and vancomycin-resistant Staphylococcus aureus.

Staphylococcus aureus, Enterobacteriaceae, Pseudomonas aeruginosa, Enterococcus, and Acinetobacter make up over half of all CABSI pathogens and are drug-resistant threats listed by the Centers for Disease Control and Prevention (CDC).

EDTA has particularly useful properties in that its antimicrobial mechanisms are not known to promote disease resistance, in contrast to prior antibiotic-based catheter locks. Further, it is effective against the “triple threat” of bacteria, biofilms, and blood clots that jeopardize so many patients receiving catheters, and its efficacy is remarkably persistent over long periods. In spite of its success, there is a need for further improvements in catheter systems and products, particularly in dealing with such situations as the transition from a less effective catheter lock solution, coping with outbreaks of drug-resistant microbes, or other challenges that may still arise from biofilms and blood clots.

While alcohol (ethanol) locks have been used and are said to be effective against many microbes, ethanol has been associated with structural changes in catheters, especially for polyurethane materials, and with problems with occlusion as well as systemic effects due to the introduction of alcohol into the bloodstream. Ethanol above a concentration of 28% can also lead to plasma protein precipitation. Further, while it has long been thought that microbes could not develop resistance to alcohol, this belief is not being questioned as researchers recently discovered that Enterococcus faecium has become as much as ten times more resistant to alcohol over a few years. This problem may prove to become much worse due to the overuse of alcohol throughout society during the COVID-era and its continued heavy use in hospitals and clinics and in the field of implantable medical devices. There is a need to continue monitoring and studying the evolving response of bacteria to alcohol and to continue strengthening systems that do not rely heavily on alcohol.

### SUMMARY

Provided herein are methods of reducing the risk of a bloodstream infection in a subject. The methods generally include removing a first lock solution from an implantable medical device; instilling a shock solution into the implantable medical device; removing the shock solution from the implantable medical device; and instilling a second lock solution into the implantable medical device, wherein the shock solution comprises ethylenediaminetetraacetic acid (EDTA) and has a pH of at least 0.2 units higher than the first lock solution, the second lock solution, or both. The methods may include instilling a shock solution into the implantable medical device; removing the shock solution from the implantable medical device; and instilling a lock solution into the implantable medical device, wherein the shock solution comprises ethylenediaminetetraacetic acid (EDTA) and has a pH of at least 0.2 units higher than the lock solution. In some embodiments, the first lock solution comprises tetrasodium EDTA at a concentration of about 4% (w/v). In some embodiments, the second lock solution comprises tetrasodium EDTA at a concentration of about 4% (w/v). In some embodiments, the shock solution further comprises an anti-biofilm agent. In some embodiments, the shock solution is instilled into the implantable medical device for a duration of at least 10 seconds. In some embodiments, the shock solution is instilled into the implantable medical device for a duration of up to 1 hour. In some embodiments, the shock solution has a pH from about 8.5 to about 12, about 9.5 to about 12, or about 10.5 to about 12. In some embodiments, the shock solution comprises EDTA having a concentration from about 4.5% to about 25% (w/v), about 8% to about 25% (w/v), or about 10% to about 25% (w/v). In some embodiments, the shock solution has a temperature from about 37.5° C. to about 90° C. In some embodiments, the implantable medical device comprises a catheter.

Further provided herein are methods of reducing the risk of a bloodstream infection in a subject. The methods generally include removing a first lock solution from an implantable medical device, the first lock solution comprising 4% ethylenediaminetetraacetic acid (EDTA) (w/v); instilling a shock solution into the implantable medical device; removing the shock solution from the implantable medical device; and instilling a second lock solution into the implantable medical device, wherein the shock solution comprises ethylenediaminetetraacetic acid (EDTA) and has a pH of at least 0.2 units higher than the lock solution.

Further provided herein are kits including a lock solution comprising 4% ethylenediaminetetraacetic acid (EDTA); and one or more shock solutions, wherein each of the one or more shock solutions comprises EDTA and has a pH of at least 0.2 units higher than the lock solution. In some embodiments, the one or more shock solutions has a pH from about 8.5 to about 11.5. In some embodiments, the one or more shock solutions comprises EDTA having a concentration from about 4.5% to about 25% (w/v). In some embodiments, the one or more shock solutions further comprises an anti-biofilm agent.

### BRIEF DESCRIPTION OF THE FIGURES

FIGS. 1A and 1B depict a catheter line with two solution sources. FIG. 1A depicts a first-filled syringe for delivery of a short-term shock treatment and a second syringe for providing a long-term lock solution while in FIG. 1B the syringe for short-term treatment also comprises a filter disk assembly comprising a soluble powder.

FIG. 2 is a flow chart describing a method of delivering a shock treatment in response to in vivo detection of biofilm growth or incipient patency reduction.

FIG. 3 depicts one version of a system for early detection of degraded implantable medical device performance due to biofilm formation or blood clot formation.

FIG. 4 depicts an EDTA-solution kit with vials of a solution, prefilled syringes for shock treatments, and MRDO-specific cartridges.

### DETAILED DESCRIPTION

The present disclosure pertains to products and methods for the improved use of an implantable medical device with respect to the threats of multidrug-resistant organisms (MDROs). The methods generally include removing a first lock solution from an implantable medical device, instilling a “shock” solution into the implantable medical device, removing the shock solution from the implantable medical device, and instilling a second lock solution into the implantable medical device. In some embodiments, the shock solution may be employed before a lock solution is ever introduced to the implantable medical device. Accordingly, in such embodiments, the methods include instilling a shock solution into the implantable medical device, removing the shock solution from the implantable medical device, and instilling a lock solution into the implantable medical device. Without wishing to be bound by theory, it is believed that the shock treatment provides a temporary elevated driving force for killing pathogens or reducing their growth, or removing related biofilm material (though the shock treatment may also be helpful for removal of blood clots or factors that can contribute to blood clot formation), wherein the shock solution has at least one of the following properties: an elevated concentration of EDTA as compared to the lock solution, an elevated pH as compared to the lock solution, an elevated temperature as compared to the lock solution, includes an anti-biofilm agent, includes an antibiotic, includes a thrombolytic agent, or includes an antiseptic agent.

The first lock solution and the second lock solution may be the same solution or may be different solutions. The lock solution generally comprises EDTA. Sodium salts of EDTA are commonly available and generally used, including di-sodium, tri-sodium, and tetra-sodium salts. However, other EDTA salts, including ammonium, di-ammonium, potassium, di-potassium, cupric di-sodium, magnesium di-sodium, ferric sodium, and combinations thereof, may be used, provided they have the anti-bacterial and/or fungicidal and/or anti-protozoan and/or anti-amoebic properties desired, and provided that they are sufficiently soluble in the solvent desired. Preferably, the lock solution comprises trisodium and/or tetrasodium EDTA. The lock solution may be terminally sterilized to ensure the safety and efficacy of the lock solution.

The lock solution may include EDTA having a concentration in the lock solution from about 1% (w/v) to about 20% (w/v). For example, the lock solution mya include EDTA having a concentration in the lock solution from about 1% (w/v) to about 4% (w/v), about 1% (w/v) to about 8% (w/v), about 1% (w/v) to about 12% (w/v), about 1% (w/v) to about 16% (w/v), about 1% (w/v) to about 20% (w/v), about 4% (w/v) to about 8% (w/v), about 4% (w/v) to about 12% (w/v), about 4% (w/v) to about 16% (w/v), about 4% (w/v) to about 20% (w/v), about 8% (w/v) to about 12% (w/v), about 8% (w/v) to about 16% (w/v), about 8% (w/v) to about 20% (w/v), about 12% (w/v) to about 16% (w/v), about 12% (w/v) to about 20% (w/v), about 16% (w/v) to about 20% (w/v), about 2% (w/v) to about 6% (w/v), about 2% (w/v) to about 5% (w/v), or about 3% (w/v) to about 5% (w/v). As another example, the lock solution may include EDTA having a concentration in the lock solution of about 1% (w/v), 1.5% (w/v), 2% (w/v), 2.5% (w/v), 3% (w/v), 3.5% (w/v), 4% (w/v), 4.5% (w/v), 5% (w/v), 5.5% (w/v), 6% (w/v), 6.5% (w/v), 7% (w/v), 7.5% (w/v), 8% (w/v), 8.5% (w/v), 9% (w/v), 9.5% (w/v), 10% (w/v), 10.5% (w/v), 11% (w/v), 11.5% (w/v), 12% (w/v), 12.5% (w/v), 13% (w/v), 13.5% (w/v), 14% (w/v), 14.5% (w/v), 15% (w/v), 15.5% (w/v), 16% (w/v), 16.5% (w/v), 17% (w/v), 17.5% (w/v), 18% (w/v), 18.5% (w/v), 19% (w/v), 19.5% (w/v), or about 20% (w/v). The lock solution may further include a thrombolytic agent, an anti-biofilm agent, an antiseptic, a buffer, or another ingredient as described further herein.

The lock solution may have a pH from about 8.5 to 11.5, such as from about 8.5 to about 9, about 8.5 to about 9.5, about 8.5 to about 10, about 8.5 to about 10.5, about 8.5 to about 11, about 8.5 to about 11.5, about 9 to about 9.5, about 9 to about 10, about 9 to about 10.5, about 9 to about 11, about 9 to about 9.5, about 9.5 to about 10, about 9.5 to about 10.5, about 9.5 to about 11, about 9.5 to about 11.5, about 10 to about 10.5, about 10 to about 11, about 10 to about 11.5, about 10.5 to about 11, about 10.5 to about 11.5, or about 11 to about 11.5. As another example, the lock solution may have a pH of about 8.5, 8.6, 8.7, 8.8, 8.9, 9.0, 9.1 9.2, 9.3, 9.4, 9.5, 9.6, 9.7, 9.8, 9.9, 10.0, 10.1, 10.2, 10.3, 10.4, 10.5, 10.6, 10.7, 10.8, 10.9, 11.0, 11.1, 11.2, 11.3, 11.4, or about 11.5. Preferably, the lock solution may have a pH such that at least 5%, 10%, 15%, 20%, or 25% of the EDTA is in the form of its tetrasodium salt.

In an example, the lock solution may include an EDTA solution for long-term use with respect to an implantable medical device comprising at least one salt of EDTA in solution, wherein the at least one EDTA salt is tri-sodium and/or tetra-sodium EDTA, at a combined tri-sodium and tetra-sodium EDTA concentration from about 1.0% (w/v) and to about 15% (w/v). The lock solution may further comprise at least 0.01% of a thrombolytic agent, such as from 0.03% to 1% (w/v) of at least one of alteplase, urokinase, and streptokinase.

In a preferred embodiment, the lock solution may include KiteLock™, which is a 4% sterile Catheter Lock Solution provided in a 3 mL volume. KiteLock™ is a single-use, clear, colourless and sterile solution. It is free of preservatives, antibiotic, alcohol and latex, and it is non-pyrogenic. KiteLock™ 4% Sterile Catheter Lock Solution is an aqueous solution containing a mixture of tetrasodium EDTA and trisodium EDTA. The solution composition is equivalent to 28 mg/mL EDTA. Each needle-free polypropylene ampoule contains 3 mL of KiteLock™ 4% Sterile Catheter Lock Solution which is equivalent to 84 mg of edetate in water for injection.

The shock solution preferably comprises EDTA. The EDTA may be present as one or more sodium salts of EDTA, including trisodium EDTA or tetrasodium EDTA. The pH of the shock solution may sufficiently high that the EDTA comprises mostly the trisodium and/or tetrasodium salt of EDTA. Preferably, the pH of the shock solution is high enough such that 5-25% (w/v) of the EDTA in the shock solution comprises tetrasodium EDTA.

The shock solution may have a concentration of EDTA in an amount of about 15% to 200% higher than the concentration of EDTA in the lock solution. As used here, the percentage refers to the relative increase of the EDTA concentration as compared to the EDTA concentration of the lock solution rather than an absolute increase of the EDTA concentration. Thus, as an illustration, if the lock solution contains EDTA at a concentration of 4% (w/v), the shock solution may have a concentration of EDTA in an amount from about 4.6% (w/v) to about 20% (w/v). For example, the shock solution may have a concentration of EDTA in an amount from about 15% to about 30% higher, about 15% to about 50% higher, about 15% to about 75% higher, about 15% to about 100% higher, about 15% to about 125% higher, about 15% to about 150% higher, about 15% to about 175% higher, about 15% to about 200% higher, about 30% to about 200% higher, about 50% to about 200% higher, about 75% to about 200% higher, about 100% to about 200% higher, about 125% to about 200%, about 150% to about 200% higher, about 175% to about 200% higher, or about 50% to about 150% higher than the concentration of EDTA in the lock solution. As another example, the shock solution may have a concentration of EDTA in an amount of about 15%, 20%, 25%, 30%, 35%, 40%, 45%, 50%, 55%, 60%, 65%, 70%, 75%, 80%, 85%, 90%, 95%, 100%, 110%, 120%, 130%, 140%, 150%, 160%, 170%, 180%, 190%, or about 200% higher than the concentration of EDTA in the lock solution.

In some embodiments, the shock solution may have a concentration of EDTA in an amount from about 15% to about 400% higher than the concentration of EDTA in the lock solution, such as from about 15% to about 250% higher, about 15% to about 300% higher, about 15% to about 350% higher, about 15% to about 400% higher, about 30% to about 400% higher, about 50% to about 400% higher, about 75% to about 400% higher, about 100% to about 400% higher, about 125% to about 400% higher, about 150% to about 400% higher, about 175% to about 400% higher, about 200% to about 400% higher, about 250% to about 400% higher, about 300% to about 400% higher, or about 350% to about 400% higher than the concentration of EDTA in the lock solution. As another example, the shock solution may have a concentration of EDTA in an amount of about 15%, 20%, 25%, 30%, 35%, 40%, 45%, 50%, 55%, 60%, 65%, 70%, 75%, 80%, 85%, 90%, 95%, 100%, 110%, 120%, 130%, 140%, 150%, 160%, 170%, 180%, 190%, 200%, 210%, 220%, 230%, 240%, 250%, 260%, 270%, 280%, 290%, 300%, 310%, 320%, 330%, 340%, 350%, 360%, 370%, 380%, 390%, or about 400% higher than the concentration of EDTA in the lock solution.

The shock solution may have a concentration of EDTA from about 4.5% to about 25% (w/v). For example, the shock may have a concentration of EDTA from about 4.5% to about 8%, about 4.5% to about 12%, about 4.5% to about 16%, about 4.5% to about 20%, about 4.5% to about 25%, about 8% to about 12%, about 8% to about 16%, about 8% to about 20%, about 8% to about 25%, about 12% to about 16%, about 12% to about 20%, about 12% to about 25%, about 16% to about 20%, about 16% to about 25%, or about 20% to about 25% (w/v). As another example, the shock solution may have a concentration of EDTA of about 4.5%, 5%, 5.5%, 6%, 6.5%, 7%, 7.5%, 8%, 8.5%, 9%, 9.5%, 10%, 10.5%, 11%, 11.5%, 12%, 12.5%, 13%, 13.5%, 14%, 14.5%, 15%, 15.5%, 16%, 16.5%, 17%, 17.5%, 18%, 18.5%, 19%, 19.5%, 20%, 20.5%, 21%, 21.5%, 22%, 22.5%, 23%, 23.5%, 24%, 24.5%, or about 25% (w/v).

The shock solution may have a pH higher than the pH of the lock solution. For example, the pH of the shock solution may be from about 0.2 to about 1.3 units higher than the lock solution. The shock solution may have a pH of about 8.5 to about 12, such as from about 9 to about 12, about 9.5 to about 12, about 10 to about 12, about 10.5 to about 12, about 11 to about 12, about 11.5 to about 12, about 9 to about 9.5, about 9 to about 10, about 9 to about 10.5, about 9 to about 11, about 9 to about 11.5, about 9.5 to about 10, about 9.5 to about 10.5, about 9.5 to about 11, about 9.5 to about 11.5, about 10 to about 10.5, about 10 to about 11, about 10 to about 11.5, about 10.5 to about 11, or about 10.5 to about 11.5. As another example, the shock solution may have a pH of about 8.5, 8.6, 8.7, 8.8, 8.9, 9.0, 9.1, 9.2, 9.3, 9.4, 9.5, 9.6, 9.7, 9.8, 9.9, 10.0, 10.1, 10.2, 10.3, 10.4, 10.5, 10.6, 10.7, 10.8, 10.9, 11.0, 11.1, 11.2, 11.3, 11.4, 11.5, 11.6, 11.7, 11.8, 11.9, or about 12.0.

The shock solution may have a temperature from about 37.5° C. to about 90° C. For example, the shock solution may have a temperature from about 37.5° C. to about 40° C., about 37.5° C. to about 50° C., about 37.5° C. to about 60° C., about 37.5° C. to about 70° C., about 37.5° C. to about 80° C., about 37.5° C. to about 90° C., about 40° C. to about 50° C., about 40° C. to about 60° C., about 40° C. to about 70° C., about 40° C. to about 80° C., about 40° C. to about 90° C., about 50° C. to about 60° C., about 50° C. to about 70° C., about 50° C. to about 80° C., about 50° C. to about 90° C., about 60° C. to about 70° C., about 60° C. to about 80° C., about 60° C. to about 90° C., about 70° C. to about 80° C., about 70° C., to about 90° C., or about 80° C. to about 90° C. As another example, the shock solution may have a temperature of about 37.5° C., 40° C., 42.5° C., 45° C., 47.5° C., 50° C., 52.5° C., 55° C., 57.5° C., 60° C., 62.5° C., 65° C., 67.5° C., 70° C., 72.5° C., 75° C., 77.5° C., 80° C., 82.5° C., 85° C., 87.5° C., or about 90° C. For a shock treatment based on temperature, a syringe or vial of the EDTA solution may be heated to the temperature described above, and then the heated solution can be used to displace the previous lock solution or other fluid in the catheter. A sufficient quantity of the heated solution may be applied to help disrupt biofilm or bacterial growth, or clear blood clot material, as needed, thereby reducing microbial threats or possibly patency of the catheter.

The shock solution may further comprise an anti-biofilm agent. The anti-biofilm agent is capable of preventing biofilm formation or reducing or eradicating an existing biofilm in the implantable medical device. Anti-biofilm agents are generally known in the art. Anti-biofilm agents for use in the shock solutions of the present disclosure include poloxamers, N-acetyl cysteine, glutathione, urea, tenzymes such as DNAse, detergents, and solvents.

In some embodiments, the anti-biofilm agent includes a poloxamer. Poloxamers are nonionic polyoxyethylene-polyoxypropylene block co-polymers. The poloxamer may include any poloxamer suitable for use in humans, such as poloxamer 188 (P188), poloxamer 388 (P388), or poloxamer 407 (also known as Pluronic® F-127).

The anti-biofilm agent may have a concentration in the shock solution from about 0.1% to about 20% (w/v) of the shock solution. For example, the anti-biofilm agent may have a concentration from about 0.1% to about 0.5%, about 0.1% to about 1%, about 0.1% to about 2.5%, about 0.1% to about 5%, about 0.1% to about 10%, about 0.1% to about 15%, about 0.1% to about 20%, about 0.5% to about 1%, about 0.5% to about 2.5%, about 0.5% to about 5%, about 0.5% to about 10%, about 0.5% to about 15%, about 0.5 to about 20%, about 1% to about 2.5%, about 1% to about 5%, about 1% to about 10%, about 1% to about 15%, about 1% to about 20%, about 2.5% to about 5%, about 2.5% to about 10%, about 2.5% to about 10%, about 2.5% to about 15%, about 2.5% to about 20%, about 5% to about 10%, about 5% to about 15%, about 5% to about 20%, about 10% to about 15%, about 10% to about 20%, or about 15% to about 20% (w/v) of the shock solution. As another example, the anti-biofilm agent may have a concentration in the shock solution of about 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, 15%, 16%, 17%, 18%, 19%, or about 20% (w/v) of the shock solution. The concentration of the anti-biofilm agent in the shock solution may be greater than the concentration of an anti-biofilm agent provided in the lock solution, such as at least 4%, 5%, 10%, 15%, 20%, 25%, 30%, 50%, or 70% greater than the concentration of the anti-biofilm agent in the lock solution.

The shock solution may further a thrombolytic agent. The thrombolytic agent may include one or more enzymes such as alteplase, streptokinase, reteplase, tenecteplase, urokinase, prourokinase, anistreplase (APSAC), etc. Alteplase can often be provided commercially as a lyophilized powder in, for example, 50 mg and 100 mg vials which may be packaged with diluent (e.g., sterile water for injection) for reconstitution. Other known thrombolytic agents may be considered if they become approved for human or animal use. Such thrombolytic agents include, for example, Desmoteplase, a highly fibrin-specific thrombolytic experimental drug. In some aspects, however, the thrombolytic agent does not include heparin or aspirin and comprises a protein or protein mixture, and more particularly comprises an enzyme or a mixture of enzymes.

The thrombolytic agent may have a concentration in the shock solution in an amount of at least about 0.01% (w/v) of the shock solution, such as from about 0.01% to about 1% (w/v). For example, the thrombolytic agent may have a concentration in the shock solution from about 0.01% to about 0.05%, about 0.01% to about 0.1%, about 0.01% to about 0.5%, about 0.01% to about 1%, about 0.05% to about 0.1%, about 0.05% to about 0.5%, about 0.05% to about 1%, about 0.1% to about 0.5%, about 0.1% to about 1%, or about 0.5% to about 1% (w/v) of the shock solution. As another example, the thrombolytic agent may have a concentration in the shock solution of about 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, or about 1% (w/v) of the shock solution. The concentration of the thrombolytic agent in the shock solution may be greater than the concentration of an thrombolytic agent provided in the lock solution, such as at least 4%, 5%, 10%, 15%, 20%, 25%, 30%, 50%, or 70% greater than the concentration of the thrombolytic agent in the lock solution.

The shock solution may further comprise an antimicrobial agent. The antimicrobial agent may include agents other than naturally occurring antibiotic drugs may be considered, such as fully synthetic non-antibacterial agents including sulfonamides and antiseptics (e.g., chlorhexidine, ethanol, etc.). The thrombolytic agent may include taurolidine, vancomycin, gentamicin, piperacillin-tazobactam, amikacin, ciprofloxacin, 5-fluorouracil, fluoroquinolones, benzalkonium chloride, teicoplanin, rifampin, miconazole/rifampicin, minocycline, minocycline/rifampin, tobramycin, ampicillin, sulbactam, fosfomycin, trimethoprim-sulfamethoxazole, sulfamethoxazole, colistin methanesulfonate (CMS), tigecycline, carbapenems, imipenem or meropenem, tobramycin, aztreonam, cefepime, linezolid, or any combination thereof. Certain combinations that may be made include colistin with aztreonam or rifampin, carbapenem plus ampicillin-sulbactam, meropenem plus an aminoglycoside, and gentamicin plus piperacillin-tazobactam.

The antimicrobial agent may have a concentration in the shock solution in an amount of at least about 0.01% (w/v) of the shock solution, such as from about 0.01% to about 1% (w/v). For example, the antimicrobial agent may have a concentration in the shock solution from about 0.01% to about 0.05%, about 0.01% to about 0.1%, about 0.01% to about 0.5%, about 0.01% to about 1%, about 0.05% to about 0.1%, about 0.05% to about 0.5%, about 0.05% to about 1%, about 0.1% to about 0.5%, about 0.1% to about 1%, or about 0.5% to about 1% (w/v) of the shock solution. As another example, the antimicrobial agent may have a concentration in the shock solution of about 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, or about 1% (w/v) of the shock solution. The concentration of the antimicrobial agent in the shock solution may be greater than the concentration of an antimicrobial agent provided in the lock solution, such as at least 4%, 5%, 10%, 15%, 20%, 25%, 30%, 50%, or 70% greater than the concentration of the antimicrobial agent in the lock solution.

The shock solution may comprise an antiseptic. The antiseptic may include ethanol, chlorhexidine, phenol, triclosan, hexachlorophene, chlorocresol, chloroxylenol, diguanides including chlorhexidine gluconate or polyhexanide (polyhexamethylene biguanide, PHMB); quinolines such as hydroxyquinolone, dequalium chloride, or chlorquinaldol; alcohols, including ethanol and 2-propanol/isopropanol; peroxides, such as hydrogen peroxide and benzoyl peroxide; iodine including povidone-iodine, tincture of iodine, or Lugol's iodine solution, including concentrations of 1% iodine or less, and quaternary ammonium cation salts such as benzalkonium chloride, cetylpyridinium chloride, cetrimide, or any combination thereof.

In some embodiments, the shock solution may include an antiseptic in a concentration of 1% or less (w/v) of the shock solution. For example, the shock solution may include an antiseptic in a concentration of 1% or less, 0.5% or less, 0.1% or less, 0.05% or less, or 0.01% or less (w/v) of the shock solution. As another example, the shock solution may include an antiseptic in a concentration of 0.01% to about 1% (w/v) of the shock solution, such as from about 0.01% to about 0.05%, about 0.01% to about 0.1%, about 0.01% to about 0.5%, about 0.01% to about 1%, about 0.05% to about 0.1%, about 0.05% to about 0.5%, about 0.05% to about 1%, about 0.1% to about 0.5%, about 0.1% to about 1%, or about 0.5% to about 1% (w/v) of the shock solution. The concentration of the antiseptic agent in the shock solution may be greater than the concentration of an antiseptic agent provided in the lock solution, such as at least 4%, 5%, 10%, 15%, 20%, 25%, 30%, 50%, or 70% greater than the concentration of the antiseptic agent in the lock solution.

In general, the shock solution may be free of or substantially free of any one or more of the compounds mentioned herein unless otherwise indicated. Thus, for example, in one aspect, the shock may comprise from 0.5 to 8% alteplase or other thrombolytic agent but at the same time may lack an effective amount of urokinase or be substantially free of urokinase, such as having less than 0.005% (w/v). With respect to enzymes, thrombolytic agents, and other agents, the solution may be considered substantially free of an agent if it is present at a level of less than 0.05% (w/v) and, more specifically, less than 0.01%, 0.005%, 0.001%, or 0.0001% (w/v). The shock solution may also be considered substantially free of an antiseptic, surfactant, or inorganic salt if it is present at less than 0.1% (w/v).

The shock solution may be instilled in the implantable medical device for a duration from about 10 seconds to about 1 hour, or, in some cases, for greater than 1 hour. Instillation of the shock solution may be accomplished rapidly, (e.g., a push over several seconds or a bolus over a few minutes), or may be accomplished slowly as a drip over several minutes to more than one hour. In some examples, the shock solution may be instilled in the implantable medical device for a duration from about 10 seconds to about 30 seconds, about 10 seconds to about 1 minute, about 10 seconds to about 5 minutes, about 10 seconds to about 10 minutes, about 10 seconds to about 20 minutes, about 10 seconds to about 30 minutes, about 10 seconds to about 45 minutes, about 10 seconds to about 1 hour, about 30 seconds to about 1 minute, about 30 seconds to about 5 minutes, about 30 seconds to about 10 minutes, about 30 seconds to about 20 minutes, about 30 seconds to about 30 minutes, about 30 seconds to about 45 minutes, about 30 seconds to about 1 hour, about 1 minute to about 5 minutes, about 1 minute to about 10 minutes, about 1 minute to about 20 minutes, about 1 minute to about 30 minutes, about 1 minute to about 45 minutes, about 1 minute to about 1 hour, about 5 minutes to about 10 minutes, about 5 minutes to about 20 minutes, about 5 minutes to about 30 minutes, about 5 minutes to about 45 minutes, about 5 minutes to about 1 hour, about 10 minutes to about 20 minutes, about 10 minutes to about 30 minutes, about 10 minutes to about 45 minutes, about 10 minutes to about 1 hour, about 20 minutes to about 30 minutes, about 20 minutes to about 45 minutes, about 20 minutes to about 1 hour, about 30 minutes to about 45 minutes, about 30 minutes to about 1 hour, or about 45 minutes to about 1 hour. As another example, the shock solution may be instilled in the implantable medical device for a duration of about 10 seconds, 20 seconds, 30 seconds, 40 seconds, 50 seconds, 1 minute, 2 minutes, 3 minutes, 4 minutes, 5 minutes, 6 minutes, 7 minutes, 8 minutes, 9 minutes, 10 minutes, 15 minutes, 20 minutes, 25 minutes, 30 minutes, 35 minutes, 40 minutes, 45 minutes, 50 minutes, 55 minutes, or about 1 hour.

In some embodiments, the shock solution or the lock solution may further include a viscosity modifier to reduce leakage from the catheter lumen while maintaining biocompatibility, a hydrophilic carrier that facilitates improved coating adherence within the catheter to enhance both antimicrobial activity and durability, a stabilizing buffer system that maintains pH balance during extended storage thereby enhancing shelf life and consistency of performance, an inorganic salt, an organic salt (e.g., sodium citrate), or a combination thereof.

In some embodiments, the method may include using two shock solutions. For example, the method may include removing a first lock solution from the implantable medical device, instilling a first shock solution into the implantable medical device, removing the first shock solution from the implantable medical device, instilling a second shock solution into the implantable medical device, removing the second shock solution from the implantable medical device, and instilling a second lock solution into the implantable medical device. In such embodiments, the first and second shock solutions may have differing pH ranges, varying concentrations of ingredients, differing temperatures, or may differ in other factors. Without wishing to be bound by theory, it is believed that the use of two distinct pH ranges in particular can, in some aspects, allow a thrombolytic agent or antimicrobial agent to be effective over a time period sufficiently long to act effectively against clots while at a pH relatively closer to physiological pH, while the EDTA can be more effective in its antimicrobial and/or anti-biofilm functions at the higher pH range, thereby allowing both compounds to have relatively optimum performance.

In some embodiments, providing two or more pH values within a single implantable medical device may include first filling a catheter line with a first shock solution comprising a thrombolytic agent at a suitable first pH, then displacing a portion of the first shock solution with a second shock solution comprising EDTA at a second pH, wherein the act of displacing may comprise flushing out some of the first shock solution or withdrawing it from a line that it is in as the second shock solution flows in to replace the removed portion of the first shock solution. In some embodiments, providing two or more pH values within a single implantable medical device may include first instilling a catheter line with a first shock solution comprising EDTA at a first pH, then displacing a portion of the first shock solution with a second shock solution comprising a thrombolytic agent at a second pH, wherein the act of displacing may comprise flushing out some of the first solution or withdrawing it from a line that it is in as the second solution flows in to replace the removed portion of the first solution.

In some embodiments, providing two or more pH values within a single implantable medical device may include instilling a shock solution at a first pH into a catheter line after first contacting a region containing a soluble material (such as a filter cartridge, discussed further below) that can increase a concentration or the pH as the solution contacts the soluble matter, such that over a period of time as the solution passes over the soluble matter the pH or concentration of an active agent in the shock solution transitions to new levels. Thus, the first pH may be near physiological pH, and after contact with the soluble matter, the pH may increase. The soluble matter may be an alkaline solid or liquid, for example. The soluble material may also or alternatively include an anti-biofilm agent, an antimicrobial agent, or a combination thereof.

In one aspect, the method further comprises the steps of (1) detecting the presence of an MDRO or an outbreak of MDRO-related infection that may pose a threat to a patient with an implantable medical device; (2) in response, applying either a prophylactic treatment if the patient is not yet known to be infected or a therapeutic treatment if the patient is suspected of being infected with an MDRO, wherein the prophylactic or therapeutic treatment comprises applying an EDTA-based shock solution to the conduit of the implantable medical device with a suitable concentration of EDTA and a suitable pH over a suitable time to be effective in reducing the growth of MDROs associated with the implantable medical device, and wherein the shock solution may also comprise one or more active ingredients selected from the group consisting of a thrombolytic agent, an anti-bacterial, an anti-biofilm agent, and an antiseptic, and wherein the pH and temperature of the shock solution when applied to a conduit of the implantable medical device may be adjusted relative to that of the previously used lock solution; and (3) monitoring the implantable medical device and the patient to determine if the treatment has been successful or if more extreme steps are taken, and if successful, using a suitable EDTA-based lock solution for long-term management of the implantable medical device. Any one of the shock solutions described above may be used in this method. When the treatment does not appear to be successful, the more extreme steps that can be taken can include the removal of the implantable medical device, which may be necessary at a relatively early stage in some cases when a patient develops a CABSI from MDROs, as well as applying stronger or different systemic antibiotics and other measures. In some aspects, the concentration of at least one compound in the shock solution, such as the EDTA or one or more of the other active ingredients, is greater than the concentration of at least one compound in the EDTA-based lock solution and may be at least 4%, 5%, 10%, 15%, 20%, 25%, 30%, 50%, or 70% greater. Methods of detecting the presence of a MDRO are generally known to those having ordinary skill in the art and are discussed further below.

In some aspects, the methods may further include identifying the specific MDROs that are posing the risk and consulting a database of information relating to EDTA-based lock solutions.

Any suitable database type may be used, such as a relational database, object-oriented database, distributed database, data warehouse, NoSQL database, and the like, including combinations thereof. Any suitable database management system may be used. Such a database may contain information based on clinical trials, laboratory testing, peer-reviewed studies, observations and empirical data from clinics and others, etc., for numerous aspects of catheter use and performance, including complications, interventions, and outcomes.

The database may include information relating to EDTA-based lock solutions. EDTA has proven effective in killing and controlling a wide variety of bacterial and fungal species. Data may therefore be compiled for easy access in a database to help inform users about the efficacy of EDTA-based products relative to numerous MDROs and other microbes, including kill times for various solutions when contacting the MDRO or other microbes, recommended concentrations and conditions, etc. Other information may be included, such as recommended frequency of flushing a catheter line, the optimum temperature to clear a microbe's biofilm matter from a catheter surface, optimum pH for biofilm or blood clot removal, the efficacy of various shock treatments, synergies against specific bacteria when combined with other compounds such as antiseptics (e.g., chlorhexidine or ethanol), thrombolytics such as alteplase, anti-fouling or anti-biofilm agents such as urea, poloxamer, or N-acetyl cysteine (NAC), antibiotics such as minocycline or taurolidine, and chelants or other agents such as sodium citrate, etc., as well as surfactants that may assist in the removal of biofilm or clearing occlusions.

Other relevant information may also be present in the database or accessible by the database, such as potential interactions with other tools, mechanisms, or treatments, such as ultrasonic clearing methods, ultrasonic activation of a surface to hinder bacterial attachment or biofilm growth or clot formation, bioelectric effects wherein a small amount of current is steadily or periodically applied to a surface to hinder microbial growth, biofilm formation, or possibly clot formation, etc. Providing such information in a database coupled with suitable tools for searching and applying database information (e.g., a useful user-interface or intelligent agents such as an AI system or machine learning tool) may be used to guide treatment of a patient with an implantable medical device to reduce the risks of MDRO infection or to swiftly respond to any incipient infection to help prevent or overcome CABSIs.

In one aspect, an ultrasonic Doppler flow analysis and visualization system may be used to measure flow in the catheter or at the catheter tip, which can be combined with dynamic pressure information in the catheter line during controlled steady or dynamic flow, including pulsatile flow (e.g., pressure pulses occurring a frequency of, for example, from 0.1 to 20 Hz). Visualization of the flow and analysis of the flow coupled with measurement of the instilled flow rate and the corresponding pressure response in the line can be combined to yield information about catheter characteristics and behavior that, in some cases, can help identity not only a change in catheter patency, but also help identify the likely nature of the change due to the different flow occlusion characteristics of biofilm, blood clots, precipitated medication, and other potential sources of occlusion. This and related flow characterization tools may provide data that can be analyzed and considered by a catheter management system, which may also comprise a fluid flow database to help correlate observed flow dynamics behavior with various catheter states. Results obtained from a patient, including behavior in response to various outcomes and analysis of a catheter once it is removed, can provide data that can be added to the database to improve machine learning related to fluid flow.

In cases when a patient already has an MDRO infection and needs a catheter, a kit may be provided with a suitable EDTA-based lock solution (e.g., solution of 4% EDTA at a pH of about 9.5) and one or more shock solutions coupled with antimicrobial wipes and other tools for sterilizing surfaces, skin, etc., to safely insert the catheter. Each of the one or more shock solutions may be provided in a pre-filled syringe (e.g., 10 mL syringes or smaller). The catheter may initially be filled with a shock solution having relatively high antimicrobial efficacy. Any of the shock solutions described hereinabove may be used. In an example, the shock solution may be an EDTA solution having a concentration of 4%, 5%, 6%, 7%, 8%, or 10% (w/v) or higher, and a pH greater than or equal to 9, 9.5, 10, 10.5, or 11.

In cases when a patient has been using a lock solution other than an EDTA-based lock solution, a transition process may be needed as the lock solution is changed to a more effective long-term strategy. To reduce the risk of bacterial infection and remove biofilm matter or blood clot material from an implantable medical device that has been managed previously without the benefits of an EDTA-based lock solution, the transition process may comprise first applying an EDTA-based shock treatment followed by using an EDTA lock solution that may be more suitable for long term locking of the implantable medical device. Any of the shock solutions or lock solutions described hereinabove may be used.

In some examples, the shock solution may have an elevated concentration (e.g., 4-10% (w/v)) of EDTA, and may be followed by providing a lock solution having a lower EDTA concentration suitable for long-term use, such as 4% (w/v) or less.

In some examples, the shock solution may have an elevated pH (e.g., 11.0), and may be followed by providing a lock solution having a lower pH (e.g., 9-10.9) suitable for long-term use.

In some examples, the shock solution may have an elevated temperature (37.5° C. and 90° C.), followed by providing a lock solution at a lower temperature (e.g., physiologic temperature).

In some examples, the shock solution may be an EDTA-based shock solution comprising at least 4% EDTA or from 4% to 25% EDTA or from 8% to 20% EDTA at a pH of at least 9.5 and further comprising an anti-biofilm agent such as a poloxamer compound (e.g., at least 0.1% of a poloxamer approved for human use such as P188, P388, and F127, or from 0.1% to 20%, 0.5% to 10%, or 0.5% to 5% of poloxamer); N-acetyl cysteine or its metabolite, glutathione; urea; other biocompatible biofilm disruptors including DNAse and other suitable enzymes; or detergents or solvents capable of removing biofilm matter.

In some examples, the method may include applying an initial shock solution comprising EDTA coupled with other agents at a higher concentration than in the formulation of the lock solution intended for long-term use prior to any lock solution being instilled in the implantable medical device, such as a solution with a relatively high level of an antibiotic or thrombolytic agent or agents such as citrates (e.g., sodium citrate), ethanol, etc.

In one aspect, in response to detecting a threat from MDROs for a patient with an implantable medical device using a first lock solution that has no or a relatively low concentration of EDTA, a for the method may include transitioning to a more effective EDTA-based lock solution (i.e., a second lock solution) to reduce the risk of CABSIs. As described above, the method may comprise not only providing a more suitable EDTA-based lock solution for long-term use, such as a Kitelock 4% solution, but first instilling a shock solution treatment to reduce the risk of infection from existing bacteria, especially MDROs, that may be associated with the implantable medical device or may be at risk of initiating an infection. The shock solution may be any one of the shock solutions described hereinabove.

In another aspect, when a particular MDRO has been identified, an active ingredient (i.e. a thrombolytic agent, an antimicrobial agent, an anti-biofilm agent, or an antiseptic) in addition to EDTA may be provided that is known to be useful in fighting the targeted MDRO. This active ingredient may be provided in a prefilled syringe, a vial, or may be provided in a cartridge that can be attached via a luer lock mechanism to a syringe containing EDTA solution such as the long-term EDTA lock solution or a prefilled syringe with a shock treatment solution. In this aspect, a variety of cartridges may be identified for specific microbes or for specific categories of microbes. The proper cartridge may be selected with the aid of a database for catheter care. The cartridges may resemble, in one aspect, Millex syringe filters or other syringe or “safe cone” filters or syringe-compatible filters with, for example, PTFE membranes or filter media made from glass microfiber and/or high-purity cellulose. They may be in polymeric housings made from polypropylene, polyethylene, or any suitable polymer. Thus, a filled syringe may be used to fill the sterile filter with an active soluble ingredient on the upstream (syringe) side of the filter assembly, first filling gradually with fluid to remove air as the filter is above the syringe. Vacuum assistance may also decrease the risk of residual air in the filter assembly, with vacuum pressure applied as fluid enters the filter housing. A suitable connection on the filter assembly can then be used to lock into a catheter port, hub, or tab to allow the fluid to be instilled into the catheter line or other conduit. This way, customized unit doses of actives to target specific MDROs can be applied to a catheter line to help reduce the risk of MDROs leading to CABSIs.

Alternatively, active ingredients targeted for specific MDROs can be provided in other pharmaceutically acceptable forms, such as in a vial of liquid or power, in a prefilled syringe containing liquid or without liquid, in a bottle, blister pack, or other dispensing means that can be combined with an EDTA solution to form a suitable shock solution for temporary treatment of an infection or risk of infection, followed by a return to a long-term lock solution once the adverse conditions that necessitated a shock treatment have subsided.

The use of cartridges or other systems to create a solution on demand may be considered for antimicrobials and other agents such as thrombolytics. For example, some thrombolytics, such as alteplase, may have relatively short half-lives once dissolved in an aqueous solution. For such compounds, efficacy can be preserved by keeping them dry until needed.

In another aspect, a method of improving the performance of an implantable medical device is provided, comprising, in response to detecting an adverse condition based on the presence of MDROs or an outbreak thereof in the vicinity that may put the patient at risk, and/or other risk factors such as detecting the presence of biofilm matter (particularly biofilm matter associated with MDROs), blood clot formation, indications of potentially dangerous bacterial growth, or reduced patency of a conduit in the implantable medical device, then applying an EDTA-based shock solution, wherein after application of the shock solution, the EDTA lock solution is then applied as a lock solution to the implantable medical device for a prolonged period of time. The shock solution and the lock solution may be any of those described hereinabove. Methods for detecting the presence of biofilm matter (particularly biofilm matter associated with MDROs), blood clot formation, indications of potentially dangerous bacterial growth, or reduced patency of a conduit in the implantable medical device are generally known to those having ordinary skill in the art.

In another aspect, a method is provided for tailoring the application of liquids (i.e. specific temperature, pH, EDTA concentration, or concentration of another active) in the conduit of an implantable medical device in response to detecting the presence or risk of MDROs, or indications of biofilm growth, particularly if associated with an MDRO or other adverse developments such as blood clot formation, or abnormal microbial growth on or in an implantable medical device, wherein the method comprises applying a shock solution, followed by applying an EDTA-based lock solution to the implantable medical device, the lock solution differing from the shock solution in at least one of the group consisting of a lower EDTA concentration, a lower pH, and a lower concentration of the one or more additional actives. The shock solution and the lock solution may be any of those described hereinabove.

In yet another aspect, a method is provided for identifying one or more pathogens, such as an MDRO associated with an implantable medical device in the body of a patient, and in response, adjusting the application of a lock solution or shock solution to reduce risks associated with the one or more pathogens, the method comprising application of a database for EDTA solution performance against various microbes including MDROs and information on treatment protocols in order to optimize the response, wherein the database comprises information on kill times required to kill specific microbes with various EDTA solutions and other useful information. This may be particularly important for identified multiple-drug resistant microbes or “superbugs,” where adjusting the lock solution or shock solution may help curtail risks more rapidly.

In a related aspect, a kit is provided comprising an EDTA lock solution and one or more containers such as prefilled syringes or vials of an EDTA-based shock solution for application in a push or bolus, optionally further comprising solution customization features to adjust one of pH, temperature, EDTA concentration, or concentration of another active to better fight an MDRO or other identified adverse condition. In one aspect, the solution customization feature comprises one or more cartridges, each with an active ingredient known to be effective in reducing a particular MDRO infection, wherein the cartridge may be connected to a syringe filled with EDTA solution that is then passed through the cartridge or makes contact with the contents of the cartridge to form a shock solution comprising the selected active ingredient disposed as a solute in the EDTA solution. In one such aspect, the EDTA solution passes from the syringe into the cartridge, which may be in the form of a syringe filter, and then into a conduit for the implantable medical device, thus instilling the shock solution in the implantable medical device.

Proper selection of the specific cartridge to use for a given situation and MDRO may be accomplished by consulting a database comprising EDTA-solution efficacy data versus various MDROs and other microbes, as well as medical information pertaining to the care of patients with implantable medical devices and the prevention of CABSIs. Such a database may be accessible via an app on a phone, a computer program or web page, a telephone system, an AI-guided chat page, a virtual doctor for providing medical guidance, or other expert systems that may incorporate machine learning, semantic search, AI features, and the like. Additional aspects of the database are discussed further herein.

Unless inherently contradictory, any aspect described herein may be used in combination with any other aspect. Thus, for example, a shock solution comprising taurolidine, EDTA, and urea may be provided with any of the aspects mentioned above, and elevated temperatures of, say, from 37.5° C. to 90° C. may be temporally used in flushing or locking a catheter line in conjunction with any of the aspects mentioned above.

For these aspects with elevated EDTA concentration in the shock treatment, both the shock treatment and the normal EDTA solution for long-term use (the baseline catheter lock solution) may be provided together in a kit comprising, for example, a vial or a prefilled syringe of the shock solution (elevated EDTA concentration and suitable pH) or a plurality of such prefilled syringes or vials and one or more vials or prefilled syringes of the long-term solution with a lower EDTA concentration. Further, the kit may comprise a plurality of cartridges holding unit doses (or any desired quantity) of active ingredients, such as specific antibiotics tailored to fight typical MDROs, whereby the EDTA solution instilled into the conduit of the implantable medical device can be tailored to help fight one or more identified MDROs. With the aid of the kit, a potential threat from an MDRO can be more effectively resisted. Further, the transition from a first lock solution to an improved second lock solution can be achieved by applying a suitable quantity of the shock solution from the prefilled syringe and then applying the normal long-term EDTA solution after a suitable time to serve as a lock solution.

In some aspects, the methods may further comprise the application of ultrasonic waves to the implantable medical device. Thus, in combination with a shock solution or during the use of the EDTA-based lock solution, an ultrasonic treatment may comprise applying ultrasonic waves by one or more transducers coupled to a conduit associated with the implantable medical device such that ultrasonic guided waves are propagated along the conduit at a suitable frequency. The applied ultrasonic frequency may be effective in preventing biofilm formation or in dispersing existing material on the conduit, such as removing fibrin, blood clots, biofilm matter or sessile bacteria from portions of a catheter tube, thereby increasing patency (improving the openness of the conduit by reducing the degree of occlusion). The ultrasonic frequency may be from 25 kHz to 1000 kHz, more specifically from 30 kHz to 200 kHz, and more specifically still from 40 kHz to 150 kHz, or from 40 kHz to 120 kHz.

One or more transducers may be used, such as one, two, three, four, or five transducers. The transducers be any type known in the art, such as piezoelectric transducers. For example, the ultrasonic energy may be guided into a conduit tube or other structure at an angle from 25 degrees to 70 degrees, such as about 45 degrees. An ultrasound coupling agent may be used to better connect the transducers to the implantable medical device. Suitable energy levels can be selected to provide ultrasonic energy to critical portions of the implantable medical device (e.g., the tip of a cannula or the wall of the conduit near the entry of the catheter into a blood vessel) that are effective at the limited deposition of microbes while not causing damage to human tissue (e.g., combined transducer output power levels of from 0.1 to 10 W, such as from 0.1 to 6 W or from 0.5 to 6 W). Ultrasonic visualization systems may be used to confirm the application of ultrasonic energy to targeted portions of the catheter inside the body and to adjust the tuning of the system (frequency, application angle, power levels, etc.) to achieve desired performance.

In another aspect, an implantable medical device such as a venous catheter comprises an ultrasound assembly with a movable ultrasound delivery member that can be activated to remove unwanted material (biofilm, fibrin, or blood clots) from the implantable medical device. In one aspect, the ultrasound assembly can be inserted into a central venous catheter with an ultrasound delivery unit mounted on a movable support structure that can be used to apply ultrasonic energy to targeted regions of the catheter. In this or other aspects involving ultrasonic energy, thrombolytic agents or antimicrobial agents, or anti-biofilm agents (e.g., N-acetyl cysteine, glutathione, or suitable enzymes such as DNAse) may be applied before or during the application of ultrasonic energy to help clear clots or biofilm and reduce the size of any released clumps of liberated matter.

In some aspects, in response to the suspected presence of MDROs, the shock solution may be provided with a gradient effect, such that a parameter like temperature, pH, or concentration of an agent changes over time as the implantable medical device receives the shock treatment, or as a conduit such as a catheter line receives an injected volume of fluid. For example, the shock solution being provided into a conduit may pass through a filter that also delivers a dissolved agent such that the concentration of the dissolved agent varies over time, such as rising to a high value initially and then steadily declining or slowly rising to a maximum level. In this way, there may be a relatively brief peak in pH or concentration of an active ingredient as part of the shock treatment, while the entire shock treatment persists over a much longer time than the time at which the peak was present. Similar concepts may apply to the use of temperature, with the temperature at the tip of the catheter or other crucial regions changing with time as the heated shock solution enters the catheter line and interacts with the line and the surrounding environment. Temperature and other parameters may also change in response to two or more liquid sources being fed into a conduit, such as a portion of liquid injected by a highly heated prefilled syringe and another portion of the liquid from a prefilled syringe at a lower temperature. When two or more sources of shock solution are being fed into a conduit, there may be a series of peaks or valleys in temperature, pH, or concentration occurring during the shock treatment, which together may have a synergistic effect on undermining a biofilm, removing or preventing blood clots, or inhibiting bacterial growth.

In light of the treatments described herein, in one aspect, a method is provided for reducing the risk of CABSIs by MDROs or other organisms and improving the state of an implantable medical device already inserted into the body of a patient (e.g., a human or an animal such as a pet or farm animal). Improving the state of the implantable medical device such as a catheter may include reducing microbes or biofilm matter in or on the implantable medical device, as well as reducing occlusion of a catheter or other flow line associated with the implantable medical device) by, for example, decreasing or hindering the growth of biofilm matter, decreasing bacterial growth associated with the implantable medical device, or reducing the existence of blood clots or factors on portions of the catheter that may promote clotting, such as fibrin or biofilm matter. The treatment to improve the state of the implantable medical device may include providing a shock solution or a series of shock solutions as described herein to flush the existing implantable medical device or associated lines thereof. In the case of transitioning from an inferior lock solution to one of the more effective EDTA-based solutions described herein, while good results may sometimes be obtained by virtue of the improved lock solution alone, in some cases, one or more shock treatments may be needed to adequately increase patency or otherwise improve the state of the catheter system.

In some aspects, the shock treatment may be provided in response to the detection of a threat, such as detection of MDROs or early detection of decreased patency (or the incipient risk of decreased patency) as biofilm matter or blood clots form, or detection or indications of other potential risk factors such as the presence or suspected presence of a multidrug-resistant microbe or indications of abnormal microbial growth (e.g., fungal infection or amoebic infection, particularly Acanthamoeba infection) that may require elevated measures to stop microbial growth on the implantable medical device (e.g., thrombolytic agents, anti-microbials, anti-biofilms, or anti-fouling agents).

Methods for detecting the presence of an MDRO are now described. Numerous factors can point to possible microbial infections or unwanted microbial growth that may indicate a need for possible shock treatment to reduce microbial growth on or associated with the implantable medical device. For example, the onset of fever, pain, or inflammation may be such an indicator. Leukocytosis, which can be detected in routine analysis of withdrawn blood, may also indicate the presence of high levels of microbes. Blood samples can also be analyzed for the presence of microbes with several methods, such as conventional culture methods or next-generation sequencing (NGS), such as versions targeting 16S rDNA, which can be PCR-amplified directly from, for example, the tip of a venous catheter (CVC) to identify pathogens associated with catheter-related infections (CRI).

By taking blood samples or, when possible, samples of material taken from the surface of the catheter line, molecular methods for detecting microbes may also be used instead of culture methods. For example, material from the catheter tip or regions comprising biofilm may be extracted with miniature collection devices that encapsulate material, which can then be analyzed using polymerase chain reaction (PCR) methods to amplify and detect microbial DNA. More generally, detection routes may include biochemical testing, nucleic acid testing, or immunoanalysis. Immunosensors can be used for indirect testing by detecting antigens (e.g., proteins or polysaccharides) from the microbes or antibodies produced by the host in response to the presence of specific microbes. Lab-on-a-chip (LOC) technology may be employed. In one aspect, target microbes in samples can be attracted by specific antibodies on microspheres or nanoparticles and then detected by immunofluorescence or other technologies. In one aspect, the target cells are captured by the microspheres or other particles marked with carbon dots that have strong fluorescence intensity during immunofluorescence testing.

In some cases, rapid staining results can be useful for initial steps because knowing if the bacteria present are Gram-positive or negative sometimes can be useful in adjusting a catheter, for some formulations or options may be more useful against Gram-positive than Gram-negative bacteria in general, or vice versa.

In response to detected microbes, datasets describing the performance of EDTA solutions and shock solutions can then be consulted to determine appropriate steps to mitigate the risk. Such data can include clinical trials examining EDTA solution performance with a wide variety of microbes. For example, it is known that a 4% EDTA solution such as Kitelock® 4% can be extremely effective as a lock solution against both Gram-positive and Gram-negative bacteria such as Staphylococcus epidermidis, Staphylococcus aureus, Pseudomonas aeruginosa, Klebsiella pneumoniae, Bacillus species, Corynebacterium species, Ralstonia insidiosa, Stenotrophomonas maltophilia, Enterobacter agglomerans, Proteus mirabilis, Enterococcus faecalis, Serratia marcescens, and Escherichia coli, and are effective in limiting the biofilm produced by such organisms. Good results have also been obtained with methicillin-resistant S. aureus (MRSA), vancomycin-resistant Enterococcus, Candida glabrata, and Candida albicans.

When the presence of such microbes is detected, such as in a catheter line that does not yet have a highly effective EDTA lock solution such as Kitelock® 4% lock solution, medical staff can then consult data that may be provided or associated with the solutions, methods, and kits described herein, to determine the required kill times for such microbes using the EDTA lock solution or one or more available alternate solutions such as a shock treatment solution. In one aspect, an app or computer program is provided that automatically evaluates the various options that can be delivered with the available kits or materials in order to recommend a catheter regimen to effectively manage the catheter to reduce the risk of infection, occlusion, or other problems.

For example, in response to indications that a hospital may have an outbreak of MRSA, protocols with catheters may be adjusted to optimize efficacy in controlling MRSA. Adjustment of pH or temperature cycles may be considered as well as the use of shock formulations with higher concentrations of various agents relative to the standard lock solution.

The formulations and methods of the present disclosure are generally safe for human and animal administration and are biocompatible and non-corrosive. They may also have anticoagulant properties and are thus useful for preventing and/or treating a variety of catheter-related infections, and may have numerous applications, including applications such as lock and lock flush solutions for various types of catheters, use as antiseptic agents or solutions for sanitizing a range of medical, dental and veterinary devices, instruments and other objects, surfaces, and the like. They furthermore may have sanitizing applications in industrial and food preparation and handling settings.

One noteworthy advantage of the EDTA-based approach described herein is that the EDTA solution itself does not pose systemic risks to the patient, and thus, there is no need to remove the lock solution from a catheter before administering medication, nutrition, fluids, or other materials to the patient. This is especially important because it means there is typically no need to introduce blood into a line used to deliver materials to the patient, and thus there is reduced contact of blood with foreign surfaces and reduced risk of materials in the blood being deposited inside the line. This can reduce infection, reduce biofilm, maintain good patency, etc. Thus, in one aspect, a method for catheter maintenance is provided that can not only provide therapeutic treatments to prevent or remove biofilm or microbial infections but can do so in a way that does not require withdrawing the lock solution from the catheter and displacing it with blood, but can generally maintain a catheter for a prolonged period of time without pulling blood into the line.

### Early Detection of Decreased Patency and Treatments in Response

While shock treatments can be helpful in reducing serious occlusion, it can be desirable to have early detection of incipient clots or biofilm matter that can then be more easily addressed with additional measures such as a shock treatment as described above or a relatively milder version of one or more of the shock treatment strategies. Many benefits can be achieved by providing early detection of factors threatening to reduce patency which can be combined with suitable variations of shock treatments described herein or simply providing more effective long-term lock solutions as described herein.

Detection of poor performance due to biofilm growth or blood clot formation in a catheter has traditionally been sensed by skilled medical staff as they sense increased pressure drop in a catheter line while trying to move fluid through the line, such as while seeking to deliver material into the bloodstream or to withdraw blood. Alert staff can detect partial occlusions due to increased resistance in the line, while full occlusions are much easier to recognize due to the inability of fluid to flow under applied pressure.

Thus, in one aspect, a method is provided for detecting a drop in patency (or a rise in occlusion) above a predetermined threshold in a catheter (e.g., at an early stage of biofilm growth) and, in response to detecting the drop in patency, applying a shock solution as described above to quickly improve patency.

Early detection or automatic detection of biofilm matter or clots on an implantable medical device such as a VAD may be achieved by optical means associated with target regions such as the cannula of a catheter. Fiber optic lines or suitable polymers forming the catheter may serve as means for transmitting light that can then be used to monitor the surface characteristics of the catheter. For example, long-period grating (LPG) optical fiber sensors may be incorporated into the lumen, walls, or outer surfaces of catheter lines to monitor biofilm formation. Optical coherence tomography (OCT), surface plasmon resonance imaging (SPRi), and other techniques may also be considered when appropriate. The use of plasmonic fiber optic sensors, for example, may provide a useful way to detect biofilm development in vivo. Electroactive biofilms may be detected via electrochemical surface plasmon resonance (EC-SPR), in which biofilm formation on a fiber optic surface shifts the refractive index. In some aspects, a gold-coated or other coated fiber optic sensor can detect biofilm formation using fiber-optic evanescence wave spectroscopy. Such fibers may have a U-shaped chemically etched region to serve as the exposed sensing region, wherein the etch into the unclad fiber sensing region increases the evanescent field intensities, and a significant decrease in transmission intensity can occur when a biofilm is formed. Optoelectronic devices, such as photodiodes, can also be applied to determine biofilm thickness. Other sensor techniques that may be considered include impedance microbiology, including electrochemical double layer (EDL) impedance and interdigitated microelectrodes (IDEs) for sensing biofilm formation via impedance, or other impedance-based microsensor systems.

Mechanical means may also be useful, such as miniature pressure sensing devices associated with the catheter line that can create an electrical signal in response to pressure changes during installation. For example, the characteristic of the pressure rise curve detected in the body of a syringe may provide data that can be analyzed to interpret what the pressure rise data reveal about the risk of occlusion and the status of biofilm or blood clots.

### Kits for Tailored Compositions

In another aspect that may be useful in fighting specific MDROs, a kit comprising an EDTA lock solution and two or more distinct shock solutions is provided that further contains modules for adjusting the formulation of the lock solution or shock solution in response to the presence of MRDOs or other threats being mitigated. Any one of the lock solutions or shock solutions described hereinabove may be used. In one aspect of this approach, a desired quantity of a material, such as an antibiotic or thrombolytic agent, normally not stable in a liquid solution, may be added to make a fresh solution easy for immediate use in a catheter line. In one case, the module may be a dry, sterile syringe or a sterile vial already comprising a soluble active material such as alteplase powder or other thrombolytic, antimicrobial, or antibiofilm agents, which may then be combined with a quantity of the lock solution or a shock solution to create a fresh shock solution comprising the desired material. A first example of a kit for MDRO mitigation is designed for managing catheter-related bloodstream infections and includes an EDTA lock solution preloaded in syringes to chelate biofilm-forming metals, along with two or more shock treatment modules: one containing a high-dose antibiotic (such as daptomycin or tigecycline) and another containing a non-antibiotic agent like antimicrobial peptides or bacteriophages. The kit also may also include a diagnostic module, such as a colorimetric test or PCR assay, to identify resistance genes (e.g., blaNDM, vanA). The kit may also include a formulation adjustment module with pH buffers or ion modifiers to tailor the lock solution based on the detected MDRO.

In an embodiment, the lock solution is supplied in a dual-chamber ampoule that separates components until activation, ensuring maximum potency upon administration. In another embodiment, the solution incorporates an oxygen scavenger that minimizes oxidative degradation, preserving the integrity of the formulation during use. In yet another embodiment, the lock solution is provided in one or more ampoules and one or more shock solutions are provided in syringes, together with antimicrobial wipes, sterile disposables, and bar-coded labels, thereby enabling immediate transition therapy and improved compliance and traceability in clinical practice.

In another embodiment, the lock solution is provided in ready-to-use prefilled syringes with tamper-evident seals to improve ease of use and safety in clinical practice.

In another embodiment, the kit includes a cartridge system is configured to provide unit doses of active ingredients targeted for specific pathogens, wherein a syringe containing EDTA solution is passed through the cartridge to create an on-demand combination lock solution adapted to combat the identified microbial threat.

A second example is a wound infection treatment kit for carbapenem-resistant Enterobacteriaceae (CRE), which provides an EDTA-based topical gel, a shock module generating reactive oxygen species (ROS), and a second shock module with bacteriophage-infused hydrogel. This kit also contains rapid diagnostic tools for resistance marker detection and formulation customization components (e.g., surfactants or gel thickeners) to adapt the treatment to wound conditions and organism profiles.

In another embodiment, one or more modules in the kit may comprise sterile cartridges such as a filter cartridge that can be attached to the tip of a syringe by a luer lock mechanism such that the fluid pushed out of the syringe flows into the filter cartridge, where a quantity of soluble powder such as alteplase powder is present on the upstream side of the filter, which can combine with the fluid from the syringe to provide a solution with the freshly dissolved solute as the fluid passes through the cartridge. The cartridge may be initially filled with ethanol, or other biocompatible fluid in which the powder to be delivered is not soluble or may comprise an airlock and air escape vent to allow initially present air in the cartridge to be expelled into the environment or into a vacuum line as fluid fills the cartridge to prevent air from entering a catheter line.

In an example, the kit may comprise cartridges with more than one formulation (e.g., differing in the type or quantity of the active), such as dry syringes filled with alteplase powder only in one aspect, and a mix of a NAC, sodium citrate, antimicrobials, and urea in another aspect, allowing the shock treatment to be customized based on the nature of the concern.

In one aspect, antiseptic compositions consisting of, consisting essentially of, or comprising one or more sodium salt(s) of EDTA at a pH higher than physiological pH are provided as antiseptic compositions of the present invention. Such antiseptic compositions have applications as lock solutions and lock flush solutions for various types of in-dwelling access catheters, including vascular catheters used for the delivery of fluids, blood products, drugs, nutrition, withdrawal of fluids or blood, dialysis, monitoring of patient conditions, and the like. Antiseptic solutions of the present invention may also be used as lock and lock flush solutions for urinary catheters, nasal tubes, throat tubes, and the like. The general solution parameters described below are suitable for these purposes. In one embodiment, an antiseptic solution consisting of, consisting essentially of, or comprising one or more sodium EDTA salt(s) at a pH higher than physiological pH is provided to maintain the patency of in-dwelling intravascular access devices. Methods for sanitizing catheters and other medical tubes, such as nasal tubes, throat tubes, and the like, are also provided and involve contacting the catheter or other medical tube with a sanitizing composition of the present invention.

According to the various aspects described herein, products comprising thrombolytic agents and EDTA may be provided in the form of catheter lock solutions suitable for use in catheters as the implantable medical device. They may be considered for a wide variety of catheters, including indwelling and short-term catheters and various catheters for venous and arterial access, such as peripheral catheters, midline catheters, tunneled and non-tunneled central venous catheters, pulmonary artery catheters, implantable catheters, umbilical catheters, and the like. The solutions and compositions disclosed herein may also be adapted for use in various products for use with catheters and catheterization, such as wipes, caps for disinfecting catheter hubs or other devices, disinfectant or preparatory sprays or solutions, etc., or may be adapted for a wide variety of other medical and cleaning products such as scrubs, sprays, or wipes for disinfecting skin or hands, wound treatments, solid surface disinfectants, and cleaners, etc. The compositions may be provided in many formats, such as in wipes, solutions in bottles or other dispensing means, and as part of a kit comprising various swabs, wipes, sprays, solutions, and other items to aid in cleaning or disinfecting or catheter preparation and maintenance, etc. The solutions may also be combined with soaps or surfactants to provide cleaning materials, ointments, etc. They may be provided as a coating or other treatment applied to solid surfaces, including being embedded on a surface or provided with encapsulation for controlled release from a surface or other material, such as being applied to or combined with materials used in a catheter such as on or in tubing, hubs, caps, etc.

In another aspect, antiseptic compositions of the present invention consisting of, consisting essentially of, or comprising one or more sodium salt(s) of EDTA at a pH greater than physiological pH are provided as sanitizing solutions for medical devices such as dentures and other dental and/or orthodontic and/or periodontal devices, for contact lenses and other optical devices, for medical and veterinary instruments, devices, and the like, and as sanitizing solutions for sanitizing surfaces and objects. Methods of sanitizing such devices are also provided, the methods comprising contacting a device with antiseptic compositions of the present invention. In general, antiseptic compositions of the present invention may be used as soaking solutions for dental, orthodontic, and periodontal devices, including toothbrushes, and are also used as soaking solutions for contact lenses and other optical devices, as well as medical and veterinary instruments, devices, and the like. For these applications, antiseptic compositions of the present invention are generally formulated as solutions or are provided in a dry form which, upon introducing a suitable solvent, forms a solution.

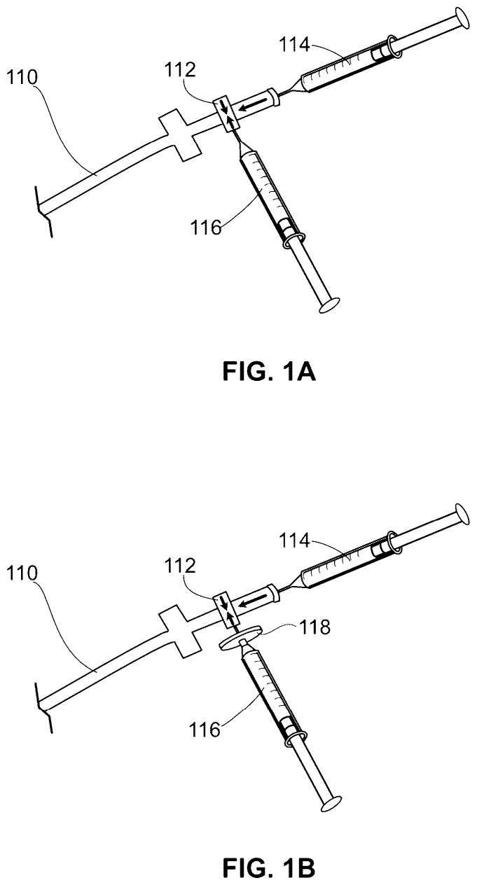

Turning now to the Figures, FIG. 1A depicts a catheter 110 connected to a three-way tap 112, which in turn is connected to two-prefilled syringes 114, 116 as described herein, a first syringe 114 containing an EDTA-based lock solution for long-term locking of the catheter 110, and a second syringe 116 containing a shock solution for the treatment of adverse conditions in the catheter 110 such as the development of biofilm matter or formation of blood clots. The second syringe 116 may include any one of the shock solutions described hereinabove.

FIG. 1B depicts a variation of the devices shown in FIG. 1A, wherein the second syringe 116 is connected via a luer lock to a cartridge 118, similar to a syringe filter assembly, wherein the cartridge 118 comprises a unit dose of a soluble powder (not shown) on the syringe-side of a filter disk (not shown) within the cartridge 118 which allows the powder to be dissolved as the solution in the syringe passes through the cartridge 118. The powder may be a particular antibiotic or a combination of anti-biofilm and antimicrobial agents such as NAC, urea, poloxamer, etc., such that passing a few milliliters of fluid (e.g., 3 to 6 ml of a 4% or higher solution of EDTA) dissolves the powder to deliver an effective dose of the solute inside the catheter. The powder may comprise a formulation such as from 5-10 parts sodium citrate, 3 parts antibiotic, 10 parts poloxamer or another surface active agent, 8 parts NAC, 8 parts urea, etc.

In an embodiment, catheter 110 may be formed from fluoropolymer composites with surface-grafted poly(ethylene glycol) chains to reduce protein adsorption, or from polyurethane blended with silver nanoparticles at 0.01-0.1% w/w for intrinsic antimicrobial protection.

In an embodiment, syringe 114 may also contain an EDTA lock solution at concentrations ranging from 2-6% with a pH of 8.5-10.2, or may be prefilled with a stabilized trisodium EDTA solution incorporating 0.01-0.05% (w/v) sodium benzoate as an antioxidant.

In an embodiment, syringe 116 may deliver a shock solution comprising 8-12% EDTA buffered with Tris at pH 10-11, or may include 0.5-2% poloxamer F127 for enhanced biofilm disruption.

In an embodiment, three-way tap 112 may be constructed from a cyclic olefin copolymer with a hydrophobic coating to minimize drug adsorption, or from a titanium-coated polymer to withstand repeated sterilization cycles.

In an embodiment, cartridge 118 may house a lyophilized thrombolytic such as alteplase (0.5-1 mg per dose) that dissolves on passage of EDTA solution, or may contain 10-50 mg of N-acetyl-cysteine in dry crystalline form for on-demand preparation.

In an embodiment, the filter disk within cartridge 118 may be fabricated from glass microfiber supporting pore sizes of 0.1-0.5μm, or from PTFE membranes coated with hydrophilic polymers for enhanced dissolution.

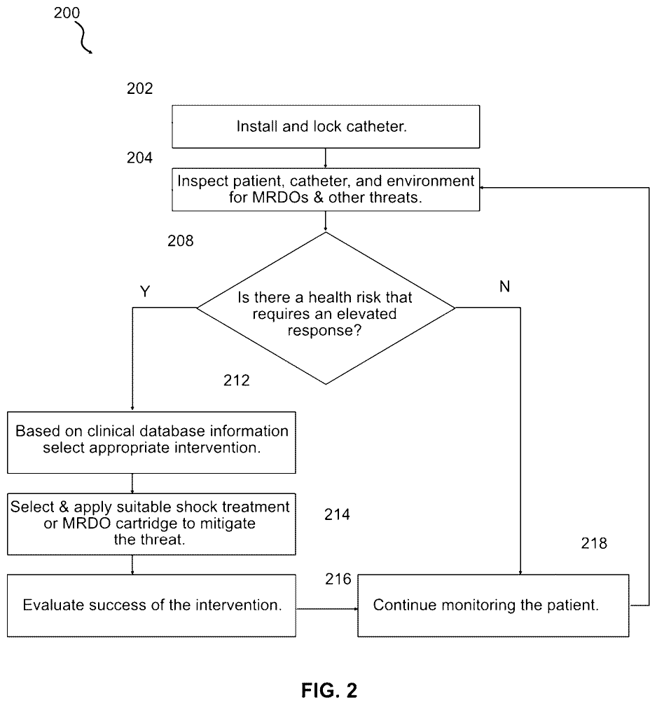

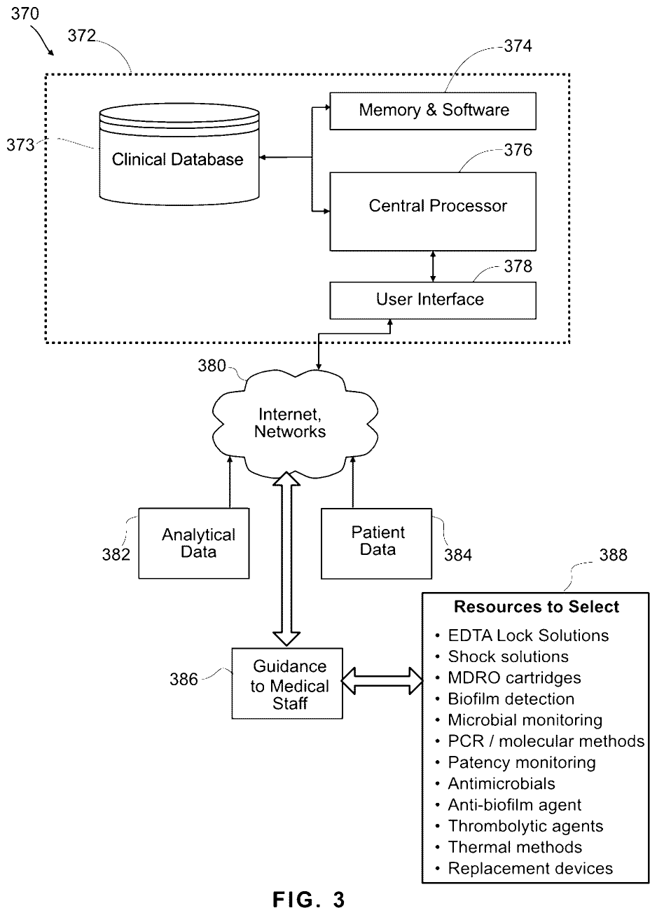

FIG. 2 is a flow chart for a method 200 of managing a catheter comprising a step 202 of installing and locking a catheter, which may include any known catheter system and may include the use of an EDTA lock solution or another lock solution. Step 204 includes inspecting the patient, the catheter, and the environment to assess threats such as the presence of MRDOs or other adverse indications of the development of biofilm matter or blood clots in the catheter. In some embodiments, step 204 may include real-time impedance microbiology using interdigitated electrodes, or fluorescence-based detection of microbial metabolites such as NADH. Evidence of elevated microbial levels is also considered in this step. Based on the observations or data from step 204, a decision is made, preferably by a healthcare worker such as a doctor or nurse, in step 208: is there a health risk that requires an elevated response? If not, the method 200 proceeds to step 218 in which the patient (and the catheter system) is monitored. If instead it is determined that there is a need for an elevated response, the method 200 proceeds to step 212 in which an appropriate intervention that may be selected based on data for EDTA solutions and other data sources. An app or computer program (not shown) may be consulted to get automated guidance via, for example, the catheter management system 370 of FIG. 3. This guidance may direct the healthcare workers to select an available shock treatment provided with a kit associated with the lock solution or as part of a kit for upgrading from an inferior lock solution to a more effective one comprising EDTA. Once the appropriate intervention is selected according to step 212, the recommended shock treatment or other intervention is applied in step 214 to mitigate the threat. The success of the intervention is then evaluated at step 216, after which necessary or recommended adjustments can be made, followed by step 218 in which the catheter and the patient are continued to be monitored.

In an embodiment, step 208 may employ Bayesian AI inference trained on 10,000+ catheter infection cases, or a rule-based system integrating pH and flow data to trigger shock therapy recommendations.

FIG. 3 depicts a catheter management system 370 for delivering guidance to medical staff 386 regarding the available resources 388 provided for the care of patients with catheters. The guidance may be provided over mobile networks or the Internet 380, responsive to analytical data 382 and patient data 384 provided from a health care facility to a remote automated system 372 comprising a central processor 376, memory and software 374, and a clinical database 373 with, for example, extensive data on the performance of catheter lock solutions and information on interventions and their success when complications arise, data on bacterial or other microbial species and associated kill times for EDTA solutions or MICs for other actives, etc. The automated system 372 may comprise AI characteristics or machine learning tools and be adapted to consider wide-ranging sources of medical and other data to supplement decision-making. It may also provide access, when needed, to human experts (not shown) that can moderate guidance.

The resources 388 may include a variety of items provided in kits according to the disclosure herein, such as EDTA lock solutions, EDTA-based shock solutions or other suitable shock solutions, and MRDO-specific cartridges. Tools such as test devices may be available for biofilm detection and monitoring microbial growth and species, including using PCR of molecular detection methods to trace bacteria in addition to conventional culture methods (not shown) or including immunoassay strips for detecting antigens associated with specific MRDOs. Further, tools may be available for detecting subtle changes in patency of catheters or other conduits, chemical or pharmaceutical agents for intervention such as anti-microbials, anti-biofilm agents, thrombolytic agents, tools for therapeutic heating (thermal methods) of catheter fluids or catheter components, and replacement parts or devices when fouling or other problems cannot be readily solved with other interventions.

In another embodiment, catheter management system 370 may integrate long-period fiber grating sensors to detect biofilm-induced refractive index changes, or surface plasmon resonance chips embedded in catheter hubs to detect microbial attachment in real-time.

In another embodiment, processor 376 may utilize edge AI hardware (e.g., FPGA accelerators) for sub-second analysis of flow/pressure data, or cloud-connected platforms with federated learning across multiple hospitals. In one example, a medical worker faced with a problem or concern in managing a catheterized patient may pose a query to the automated system 372 and provide related observations (patient data 384) and analytical data 382 if not already automatically available, and then receive guidance 386 in return. The guidance may recommend, for example, how to adjust a customizable shock treatment solution, how much to deliver, at what speed (e.g., push, bolus, or drip), and what other steps to take. Results from the intervention can then be provided as patient data 384 to enhance the learning of an AI or a machine learning system, or at least to further enrich the clinical database 373.

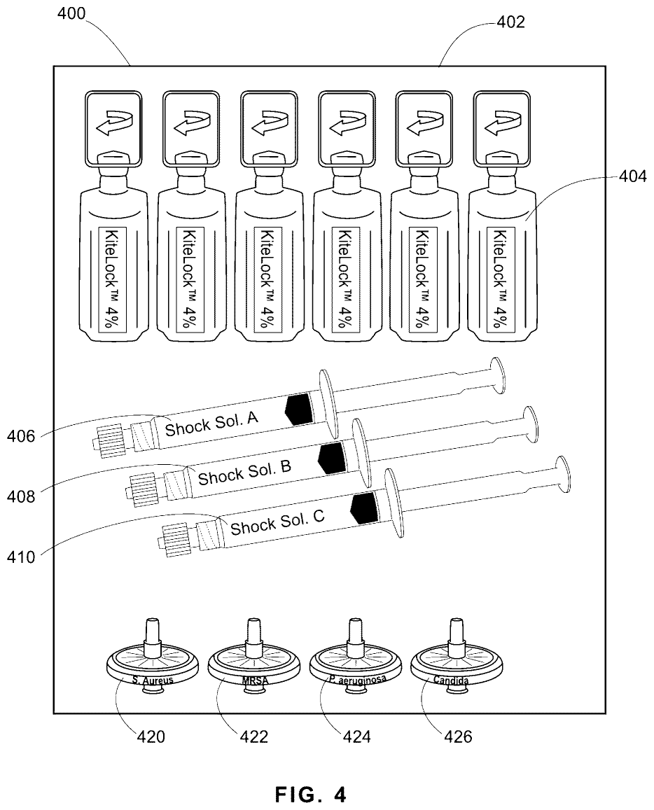

FIG. 4 depicts an EDTA-solution kit 400 provided in a container 410, such as a box or flexible pouch. The kit 400 comprises multiple vials of EDTA solution 404 and several prefilled syringes for shock treatments 406 and 408. 410, and several MDRO-specific cartridges 420, 422, and 424 for delivering additional actives needed to cope with specific MDROs. Prefilled syringe 406 is labeled “shock treatment A” and is intended for a specific scenario, such as transitioning from a non-EDTA solution to an EDTA-based lock solution, such as the vials of EDTA solution 404 in the kit 410. Prefilled syringe 408 is labeled “shock treatment B” and may be adapted for a second scenario, such as reducing biofilm matter after prolonged use. Prefilled syringe 410 is labeled “shock treatment C” and may be adapted for a third scenario, such as coping with increased blood clotting or reduced patency of the catheter (not shown). MDRO-specific cartridge 420 is labeled for S. Aureus, while MDRO-specific cartridge 422 is labeled for MRSA, MDRO-specific cartridge 424 is labeled for P. aeruginosa, and MDRO-specific cartridge 426 is labeled for Candida species (such as those documented in [0070]). Each may contain specific material suitable for preventing or reducing infection by those specific microbes when additional measures are needed. The use of any such additional measures or shock treatments may be guided by consulting a clinical database, such as via the catheter management system 370 of FIG. 4.

In an embodiment, MRDO-specific cartridges 402 may include preloaded vancomycin microspheres at 10-50 μg per cartridge, or may contain dry sodium citrate/urea blends that dissolve into an EDTA solution to form a synergistic lock solution.

In an embodiment, prefilled syringes 404 may be dual-chamber syringes holding EDTA and a second thrombolytic agent (e.g., urokinase 0.5-5%), or syringes incorporating microheaters to elevate solution temperature to 40-60° C. immediately before instillation.

### Exemplary Applications

Several illustrative applications for the compositions described herein are now set forth as proposed prophetic examples. These examples are set forth as illustrative versions of the methods and products described herein and are meant to be non-limiting.

### Catheter Kit

A catheter kit is provided in a suitable sterile package, the kit comprising (1) a plurality of vials containing EDTA lock solution comprising from 3% to 15% EDTA at a pH of from 8.5 to 10.5; (2) a prefilled syringe filled with an EDTA-based shock solution having at least one of the group consisting of: (a) an EDTA concentration at least 5% higher than the concentration of the EDTA lock solution, (b) a pH at least 0.3 units greater than the pH of the lock solution, (c) an effective amount (e.g., at least 0.1% or 1%) of an anti-biofilm or antimicrobial agent such as NAC, urea, a poloxamer, or an antibiotic such as taurolidine, and (d) at least 0.1% an thrombolytic agent such as alteplase, wherein the concentration of any selected agents from (c) or (d) is at least 50% greater than the corresponding concentration of such agents in the lock solution, if present; and (3) a plurality of MRDO-specific cartridges providing specific compounds to kill or control growth of the targeted MRDO, wherein the compounds may be combined with an EDTA solution an instilled into a catheter. The kit may also comprise sterile wipes, sterile catheter tubing, a catheter hub, a disinfectant spray, and other tools and aids.

By way of example, the lock solution may be a 4% EDTA solution at a pH of about 9.5, and in one aspect, the shock solution is 8% EDTA at a pH of 9.8 with 0.3% poloxamer, 0.5% urea, and 0.3% NAC. The MRDO-specific cartridges may comprise antibiotics, each adapted individually to oppose one or more specific MRDOs, and may comprise a powder or a liquid. In another aspect, the shock solution may comprise 5% EDTA at a pH of 9.5, 0.5% taurolidine, 3% sodium citrate, and 2% alteplase. In yet another aspect, the shock solution comprises 5% EDTA at a pH of 10, 60% ethanol, and 0.3% urea.

### Shock Solutions

For long-term maintenance of a catheter in a blood vein, a lock solution is provided that is a 4% EDTA solution at a pH of about 9.5, and a shock solution is provided that comprises 8% EDTA at a pH of 9.8 with 0.3% poloxamer, 0.5% urea, and 0.3% NAC. In response to early signs of a fever that might be associated with a bacterial infection of a catheter in a setting where infection by S. aureus has occurred recently, a cartridge for S. aureus mitigation may be selected. It may be attached to a prefilled syringe containing a shock solution adapted for fighting biofilm and catheter infection in general. The shock solution is delivered through the cartridge, adding an antibiotic effective against multidrug-resistant S. aureus, and is provided in a bolus of about 4 ml over 30 minutes, followed by the application of the lock solution for at least 8 hours.

In another aspect, the shock solution may comprise 5% EDTA at a pH of 9.5, 0.5% taurolidine, 3% sodium citrate, and 2% alteplase. In yet another aspect, the shock solution comprises 5% EDTA at a pH of 10, 60% ethanol, and 0.3% urea.

### Method for Removing Biofilm in a VAD

By way of example, a Hickman central venous catheter, a tunneled catheter, is described as having a conduit adapted for use with plasmonic fiber optic sensors or surface plasmon resonance to detect early stages of biofilm growth near the catheter tip or other portions thereof. In response to early indications of deleterious biofilm formation, a shock solution is provided in the catheter, and an ultrasonic tool is applied for a period of time to provide energy to the conduit walls in order to remove the material or reduce biofilm growth. The shock solution may comprise an anti-biofilm agent, such as 8% EDTA with 1% NAC and 1% urea at a pH of 8.5-10.5. Ultrasonic energy may be applied to the conduit by a mounted transducer outside the body or by an ultrasonically active probe that is pushed into the catheter, with power provided through a wire in communication with the probe via the catheter line.

### Use of an Implantable Medical Device Management System

In one example, an automated implantable medical device management system, such as a catheter management system, is provided in which the clinical database combines two or more database types. A relational database running SQL is provided, which contains extensive medical record information about catheterized patients with data organized according to patient characteristics, treatments received, implantable medical device information, lock solutions used with extensive data for EDTA-based solutions (but optionally also data for other types of solutions such as alcohol locks, heparin locks, etc.), CABSIs data and attendant interventions taken, MRDO threats present, bacterial culture data, outcomes, physician observations and recommendations, indications of biofilm growth or blood clot formation, protocols and products used, etc. A second database with a different structure, such as a NoSQL database, may also be used to provide access to wide-ranging, less structured data from corporate reports, published articles, news stories, etc., that can be accessed by the database management system by machine learning or AI tools to augment or assist in the interpretation of data and the generation of recommendations. Thus, for example, a physician managing a patient with a VAD seeking guidance on a possible intervention for what may be an incipient MDRO infection may query the database asking for effective interventions for the adverse situation encountered, providing data on current protocols and patient status. The automated system can then provide not only directly related data from the relational database with details of suitable EDTA-solution strategies and EDTA-based shock treatments but also insights from peripheral works that may be relevant, and these insights may be extracted and interpreted with AI guidance and optionally input from expert human sources. Thus, the physician may be given guidance and expected outcomes, based on clinical data, for an EDTA-based shock treatment followed by a specific long-term EDTA-based lock solution and may receive information on alternate possibilities and treatment options if supplied information or speculation on the MDRO is incorrect, or if other causalities are at play.

As an illustration of how such a system could be used, in one aspect, a cancer patient with a VAD for receiving chemotherapy is found to have been treated by a staff member whose improper attention to the sanitary protocol may have contributed to a recent outbreak of an MRDO, Pseudomonas aeruginosa, at a clinic. Concerned about the risks to the patient, a physician seeks to elevate measures against that particular threat. Interacting with a remote catheter management system via a graphical user interface on an iPad, the physician uploads an outbreak report, details of patient treatments and recent exposure risks (including patient medical records already accessible by the catheter management system), and requests guidance on optimum prophylactic measures. An AI-based medical guidance engine interprets the patient data, outbreak data, and other factors and accesses extensive data on lock solution performance and shock treatment performance to identify optimum routes. The AI-generated report weights the risks of unnecessary antibiotic treatments and other factors against the risks of not elevating treatments and provides a report that is reviewed and approved by a medical expert associated with the remote catheter management system, and the expert, through the user interface, speaks with the physician to discuss options. The report, in this case, concludes that a temporary shock treatment of EDTA solution at an elevated concentration without added antibiotics may be the optimum approach for this particular patient.

Provided in the results are charts showing the probabilities of various outcomes based on several sets of assumptions regarding the initial state, with each state assigned probabilities as well, allowing risks and expected outcomes to be estimated. Timelines are provided showing critical expected indicia related to the various scenarios so that new observations and test results can be quickly or automatically interpreted in light of expected scenarios and treatment protocols can be adjusted.

In this case, the physician accepts the recommendation and implements the shock treatment. The next day, a slight temperature rise is detected and communicated to the physician and the catheter management system, which recommends that the temperature rise is not likely due to an infection but recommends a blood test to monitor white blood cell count and other parameters. Again, the results appear to be no cause for alarm, and the patient returns home after a successful chemotherapy session.

In another hypothetical case, a similar patient has a catheter whose electronic pressure monitoring system detects a pressure rise response indicative of biofilm formation or precipitation of medication in the catheter during installation of medication. The data is shared with the catheter management system, which in turn recommends a pressure response test using automated equipment to provide a controlled pulsatile flow coupled with monitoring of the catheter using ultrasonic Doppler anemometry to detect the velocity and direction of fluid streams as well as turbulence and other fluid dynamic parameters at the catheter tip and/or in portions of the catheter line. By providing a known profile for the volume of fluid instilled per unit time or a known pressure delivered per unit time during, for example, pulsatile, ramped, or steady fluid flow of saline or a lock solution, the response of other parameters (e.g., velocity, flow rate, pressure, pressure drop, turbulence, velocity profiles, etc.) can be interpreted to infer the nature of occlusions affecting the catheter. This can be done by an AI-driven catheter management system with access to a fluid flow database that relates various fluid flow observations and test results to the status of the catheter, which in turn can be related to information in an EDTA solution database or other clinical databases to give guidance on interventions and forecasts, and on future steps to consider as a result of outcomes of initials treatment steps. Thus, fluid flow data for the catheter can be used as part of the data considered for a catheter management system to provide automated recommendations based on machine learning and a fluid flow database associated with the computerized catheter management system that may include. Such recommendations may include carrying out steps such as (1) applying a suitable shock treatment of an EDTA solution optionally comprising a suitable thrombolytic agent, anti-biofilm agent, an anti-fouling agent such as a surfactant, heated solution, etc. ; (2) applying an ultrasonic defouling tool to remove biofilm debris in the catheter line, optionally in combination with the application of suitable anti-biofilm and anti-fouling agents to emulsify or remove biofilm or disperse precipitated medications, optionally in combination with applied thermal energy, etc.; (3) employing the bioelectric effect in which a small current or voltage is applied to a surface in the catheter to hinder microbial growth and reduce biofilm or other occlusion sources, based on data from clinical and experimental trials made available in the database accessible by the catheter management system; (4) replacing or removing the catheter, etc.

When introducing elements of aspects of the invention or the embodiments thereof, the articles “a,” “an,” “the,” and “said” are intended to mean that there are one or more of the elements and thus may include plural referents unless the context clearly dictates otherwise. The terms “comprising,” “including,” and “having” are intended to be inclusive and mean that there may be additional elements other than the listed elements.

As used herein, the word “exemplary” means serving as an example, instance, or illustration. The aspects described herein are not limiting but rather are exemplary only. It should be understood that the described aspects are not necessarily to be construed as preferred or advantageous over other aspects. Unless otherwise indicated, no aspect of any invention described herein should be assumed to have the same advantages or features had by any other aspect.

As used herein, a “subject” is a human subject.

Having described aspects of the invention in detail, it will be apparent that modifications and variations are possible without departing from the scope of aspects of the invention as defined in the appended claims. As various changes could be made in the above compositions, products, and methods without departing from the scope of aspects of the invention, it is intended that all matter contained in the above description shall be interpreted as illustrative and not in a limiting sense.

While the foregoing description references particular illustrative embodiments, these examples should not be construed as limitations. The inventive system, methods, and products can be adapted for other uses or provided in other forms not explicitly listed above and can be modified in numerous ways within the spirit of the present disclosure. Thus, the present invention is not limited to the disclosed embodiments but is to be accorded the widest scope consistent with the claims below.

What is claimed is:

1. A method of reducing the risk of a bloodstream infection in a subject comprising:
removing a first lock solution from an implantable medical device;
instilling a shock solution into the implantable medical device;
removing the shock solution from the implantable medical device; and
instilling a second lock solution into the implantable medical device,
wherein the shock solution comprises ethylenediaminetetraacetic acid (EDTA) and has a pH of at least 0.2 units higher than the first lock solution and the second lock solution.

2. The method of claim 1, wherein the first lock solution comprises tetrasodium EDTA at a concentration of about 4% (w/v).

3. The method of claim 1, wherein the second lock solution comprises tetrasodium EDTA at a concentration of about 4% (w/v).

4. The method of claim 1, wherein the shock solution further comprises an anti-biofilm agent.

5. The method of claim 3, wherein the anti-biofilm agent comprises a poloxamer compound, N-acetyl cysteine, glutathione, urea, DNAse, a thrombolytic agent, a detergent, a solvent, or any combination thereof.

6. The method of claim 1, wherein the shock solution is instilled into the implantable medical device for a duration of at least 10 seconds.

7. The method of claim 1, wherein the shock solution is instilled into the implantable medical device for a duration of up to 1 hour.

8. The method of claim 1, wherein the shock solution has a pH from about 8.5 to about 12.

9. The method of claim 1, wherein the shock solution has a pH from about 9.5 to about 12.

10. The method of claim 1, wherein the shock solution has a pH from about 10.5 to about 12.

11. The method of claim 1, wherein the shock solution comprises EDTA having a concentration from about 4.5% to about 25% (w/v).

12. The method of claim 1, wherein the shock solution comprises EDTA having a concentration from about 8% to about 25% (w/v).

13. The method of claim 1, wherein the shock solution comprises EDTA having a concentration from about 10% to about 25% (w/v).

14. The method of claim 1, wherein the shock solution has a temperature from about 37.5° C. to about 90° C.

15. The method of claim 1, wherein the implantable medical device comprises a catheter.

16. A method of reducing the risk of a bloodstream infection in a subject comprising:
removing a first lock solution from an implantable medical device, the first lock solution comprising 4% ethylenediaminetetraacetic acid (EDTA) (w/v);
instilling a shock solution into the implantable medical device;
removing the shock solution from the implantable medical device; and
instilling a second lock solution into the implantable medical device,
wherein the shock solution comprises ethylenediaminetetraacetic acid (EDTA) and has a pH of at least 0.2 units higher than the lock solution.

17. A kit comprising:
a lock solution comprising 4% ethylenediaminetetraacetic acid (EDTA); and
one or more shock solutions, wherein each of the one or more shock solutions comprises EDTA and has a pH of at least 0.2 units higher than the lock solution.

18. The kit of claim 17, wherein the one or more shock solutions has a pH from about 8.5 to about 11.5.

19. The kit of claim 17, wherein the one or more shock solutions comprises EDTA having a concentration from about 4.5% to about 25% (w/v).

20. The kit of claim 17, wherein the one or more shock solutions further comprises an anti-biofilm agent.
